(19) United States
(12) Patent Application Publication
(10) Pub. No.: US 2026/0334953 A1
(43) Pub. Date: Oct. 29, 2026

(54) DEVICE AND DELIVERY SYSTEM FOR VASCULAR OCCLUSION OF ANEURYSMS

(71) Applicant: Elevate Innovations, LLC, Warren, NJ (US)

(72) Inventors: Cesar Alberto RINCON, Easton, PA (US); Jin PARK, Morris Plains, NJ (US); Daniel OLSEN, Asbury, NJ (US); Cory ZEGEL, Hoboken, NJ (US); Andrew Frederick DUCRUET, Paradise Valley, AZ (US); Richard Webster CROWLEY, Hinsdale, IL (US)

(21) Appl. No.: 19/656,071

(22) Filed: Apr. 23, 2026

Related U.S. Application Data
(60) Provisional application No. 63/796,496, filed on Apr. 29, 2025.

(51) Int. Cl. A61B 17/12; A61B 17/00

(52) U.S. Cl. CPC A61B 17/12113; A61B 17/12168; A61B 2017/00526; A61B 2017/00778; A61B 2017/00867; A61B 2017/12054

(57) ABSTRACT

An intrasaccular implant for vascular occlusion of an aneurysm comprises a self-expanding braided mesh body configured for constrained delivery through a catheter and expansion within an aneurysm sac. The braided mesh body includes a distal loop portion configured to contact a distal wall of the aneurysm sac over a widened contact area during deployment, a proximal loop portion configured to be positioned adjacent an aneurysm neck, and an intermediate portion between the distal loop portion and the proximal loop portion. The braided mesh body is configured such that, during deployment from the catheter, the distal loop portion expands before the proximal loop portion and the braided mesh body foreshortens in stages as successive loop portions expand. The proximal loop portion defines a recessed proximal region configured to house a release mechanism such that the release mechanism does not protrude beyond a proximal end of the implant after deployment.

### TECHNICAL FIELD

The present invention is generally related to the field of intravascular therapeutic devices and delivery systems, and more specifically to intrasaccular implants and their corresponding delivery systems. More particularly, this invention is related to self-expanding mesh implantable medical devices and delivery systems for use in the neurovascular anatomy or similar vascular anatomies for treatment of aneurysms.

### BACKGROUND

Cerebral aneurysms are a current focus of innovation in the healthcare industry, as they pose an incredibly high risk to patients, with minimal to no symptoms to forecast the potential risk. Cerebral aneurysms may be formed due to a thinning or weakening of a vessel wall within the neurovasculature, resulting in a bulge or ballooning at that region. Over time, blood flow may cause aneurysms to grow in size, with the aneurysm ranging in both size and shape. Cerebral aneurysms can range from less than 0.5mm to greater than 25mm in width, and can be treated in a variety of ways, such as clipping, coiling, flow diversion, or intrasaccular implantation.

Aneurysm shapes are categorized as spherical, cylindrical, or irregular. This shape refers to the profile of the aneurysm, and the opening to the parent vessel (neck) is categorized as regular or wide, depending on the size of the neck relative to the diameter of aneurysm. In spherical aneurysms, the width and height of the aneurysm are approximately equal. With cylindrical aneurysms, the width and height of the aneurysm are not equal, and either the width or the height may be the larger of the two measurements. From an intervention perspective, aneurysms with larger width to height ratios are considered more difficult to treat, as larger necks increase the overall difficulty of the device to be properly positioned and retained within the aneurysm sac.

Cerebral aneurysms typically present as asymptomatic, and are usually found accidentally while performing unrelated tests or scans. However, while unruptured aneurysms are typically asymptomatic, aneurysms may rupture, with a mortality rate near fifty percent. Of those that survive, approximately two-thirds suffer some permanent neurological deficit. Therefore, the risk is substantial, and when discovered, cerebral aneurysms should be treated to mitigate patient risk so long as it is safe for the patient to undergo treatment.

It is estimated that two percent of people have unruptured aneurysms, and approximately 30,000 people in the United States will suffer a cerebral aneurysm rupture each year. Although the occurrence rate is low, given the substantial risk associated with ruptured aneurysms, a safe, effective, and cost-sensitive solution is necessary.

Cerebral aneurysms are currently treated using the concept of flow diversion, where blood flow is intentionally diverted away from the aneurysm to minimize the risk of rupture and expansion. Additionally, as blood flow within the aneurysm stagnates, the aneurysm should thrombose, further increasing the resistance to blood flow inside the aneurysm sac. With sufficient thrombosis, the vessel may fully heal and the risk of an aneurysm rupture can be heavily mitigated or altogether eliminated. Flow diversion may be achieved in a variety of methods, with current treatments including: clipping, where the aneurysm neck is clamped down via an open brain surgery to prevent blood flow into the aneurysm; coiling, where the interior volume of the aneurysm is filled with metal wire via intravascular techniques; flow diverters, where a stent-like mesh or braided tube is placed in the parent vessel near the neck of the aneurysm via intravascular techniques; and intrasaccular implants, where an expandable mesh is placed inside of interior of the aneurysm to create a barrier or diversion to blood flow via intravascular techniques.

Industry preference is to treat aneurysms via intravascular techniques, as this is considered safer for the patient and should decrease recovery time while simultaneously reducing hospital stays and potential complications. Across any interventional procedure, speed and ease of use are paramount. Coiling takes time, as the goal of the physician is to insert as much material as possible into the aneurysm, and flow diverters result in an implant remaining in the parent vessel, introducing potential future risk. To meet the industry needs, a device which can occlude an aneurysm quickly and easily, being isolated to the aneurysm and not the parent vessel would be optimal. Intrasaccular implants address this need. However, the current state of the art is limited and has drawbacks that have minimized wide-spread adoption.

The current art of intrasaccular implants present several key issues that need to be addressed prior to the device being the primary or preferred method of treatment. Safety and ease of use are the primary areas of innovation and attention.

The current art presents potential risks to safety devices which may introduce risk to the distal wall of the aneurysm, as when they are inserted, they may create a “spearing” effect, where the implant comes to a point on the distal end as it is advanced out of the microcatheter. It is possible that this distal end can make contact with and damage the distal wall of the aneurysm, with the ultimate risk being a rupture of the aneurysm during the procedure. Minimizing the risk of contact with the distal wall of the aneurysm is paramount, as this is typically the weakest portion of the aneurysm and, therefore, the most likely location to rupture. Having a device which may cause concentrated forces to be placed on the distal wall of the aneurysm introduces safety concerns and increases the overall difficulty of the procedure, as it will be up to the interventionalist to ensure that the device is positioned adequately to minimize this risk.

The position of the device within the aneurysm can have a significant impact on the device effectiveness. In current practice, devices may be deployed and resheathed multiple times in order to get the optimal orientation. It is not uncommon that multiple devices are used to determine the correct sizing for the aneurysm. Current practice utilizes relatively specific sized devices which must be accurately sized for effective use. However, due to the irregularity of aneurysms, accurate sizing is challenging in practice, so a device which provides more flexibility on the range of aneurysm sizes that it can treat would provide a significant benefit to the overall ease of the procedure and reduce costs associated with procedural time and additional device usage.

Therefore, there is a distinct need for a device which can be easily implanted without significant interventionalist skill, is capable of occluding the aneurysm, and provides a procedure which minimizes the risk of patient injury.

### SUMMARY

At least the above-discussed need is addressed and technical solutions are achieved in the art by various embodiments of the present invention.

In some embodiments of the invention, an intrasaccular implant for vascular occlusion of an aneurysm comprises a self-expanding braided mesh body configured for constrained delivery through a catheter and expansion within an aneurysm sac. The braided mesh body includes a distal loop portion configured to contact a distal wall of the aneurysm sac over a widened contact area during deployment, a proximal loop portion configured to be positioned adjacent an aneurysm neck, and an intermediate portion between the distal loop portion and the proximal loop portion. The braided mesh body is configured such that, during deployment from the catheter, the distal loop portion expands before the proximal loop portion and the braided mesh body foreshortens in stages as successive loop portions expand. The proximal loop portion defines a recessed proximal region configured to house a release mechanism such that the release mechanism does not protrude beyond a proximal end of the implant after deployment.

In some embodiments of the invention, the distal loop portion includes a distal loop, the middle loop portion includes a middle loop, and the proximal loop portion includes a proximal loop. The distal loop, the middle loop, and the proximal loop are connected in series by a distal neck and a middle neck.

In some embodiments of the invention, the distal loop has a single mesh layer and each of the middle loop and the proximal loop has a double mesh layer.

In some embodiments of the invention, the double mesh layer of at least one of the middle loop or the proximal loop comprises two offset layers arranged to reduce wire overlap in a cross-sectional view parallel to the aneurysm neck.

In some embodiments of the invention, the middle loop has a diameter greater than each of a diameter of the distal loop and a diameter of the proximal loop, to increase radial retention within the aneurysm sac.

In some embodiments of the invention, the middle loop has a diameter less than a diameter of the proximal loop and the proximal loop extends distally beyond a proximal end of the middle loop to increase retention in a wide-neck aneurysm.

In some embodiments of the invention, the proximal loop includes an invaginated loop that further recesses the release mechanism within the implant.

In some embodiments of the invention, a wire braid forming the braided mesh body initiates and terminates at the recessed proximal region that houses the release mechanism, such that the implant is free of a distal braid termination point.

In some embodiments of the invention, the braided mesh body includes two loops, a distal loop and a proximal loop. The proximal loop is configured to provide both radial retention within the aneurysm sac and resistance to blood flow at the aneurysm neck.

In some embodiments of the invention, a delivery system for delivering an intrasaccular implant into an aneurysm sac comprises an outer tube, a pull wire movable relative to the outer tube, a release mechanism fixed to a distal end region of the outer tube and including distal fingers having nest holes sized to receive a ball feature of the intrasaccular implant, and a sleeve coupled to the pull wire and movable relative to the distal fingers between an engaged position in which the sleeve compresses the distal fingers to retain the ball feature in the nest holes and a disengaged position in which the sleeve is retracted to permit the distal fingers to release the ball feature.

In some embodiments of the invention, the release mechanism further includes proximal fingers having finger tabs received in slots of the outer tube and a proximal finger collar disposed over the finger tabs to lock the release mechanism to the outer tube.

In some embodiments of the invention, the release mechanism includes a flexible pattern between the distal fingers and proximal fingers to increase flexibility of the delivery system.

In some embodiments of the invention, the distal fingers are formed of a shape memory alloy and are biased toward a splayed configuration when the sleeve is in the disengaged position.

In some embodiments of the invention, the release mechanism comprises three distal fingers and the sleeve comprises three sleeve fingers nested with the distal fingers.

In some embodiments of the invention, a distance between the nest holes and distal ends of the distal fingers is minimized to increase angular articulation between the delivery system and the intrasaccular implant while the intrasaccular implant remains engaged to the delivery system.

In some embodiments of the invention, a method of manufacturing an intrasaccular implant, comprises braiding a plurality of wires to form a braided mesh body, forming the braided mesh body into a distal loop, a middle loop, a proximal loop, a distal neck between the distal loop and the middle loop, a middle neck between the middle loop and the proximal loop, and an internal proximal loop recessed within the proximal loop, and securing proximal ends of the plurality of wires at a device release mechanism coupled to the internal proximal loop such that the plurality of wires initiates and terminates at a proximal end of the intrasaccular implant.

In some embodiments of the invention, forming the braided mesh body comprises forming the distal loop as a single-layer mesh and forming each of the middle loop and the proximal loop as a double-layer mesh.

In some embodiments of the invention, forming at least one of the middle loop or the proximal loop as the double-layer mesh comprises offsetting two wire layers relative to one another to reduce wire overlap in a cross-sectional planar view parallel to an aneurysm neck.

In some embodiments of the invention, braiding the plurality of wires comprises braiding from 24 to 96 wires, the wires having diameters ranging from 0.0008 inches to 0.0012 inches.

In some embodiments of the invention, the method further comprises assembling the device release mechanism by adhering a collar, an inner collar, and terminations of both ends of the braided mesh body to each other by welding, adhesive, UV curing, or compression fitting, and coupling a tail comprising a proximal ball, a distal ball, and a shaft extending therebetween to the device release mechanism such that the shaft extends through the inner collar and the proximal ball and the distal ball are retained against removal from the inner collar.

These and other embodiments of the invention are discussed in detail below.

### BRIEF DESCRIPTION OF THE DRAWINGS

It is to be understood that the attached drawings are for purposes of illustrating aspects of various embodiments and may include elements that are not to scale. It is noted that like reference characters in different figures refer to the same objects.

FIG. 1A depicts a cross section of the intrasaccular implant in the deployed configuration with a mechanical release feature, according to an embodiment of the invention.

FIG. 1B depicts a section of the isometric view of the intrasaccular implant in the deployed configuration with a mechanical release feature, according to an embodiment of the invention.

FIG. 2A depicts the intrasaccular implant from FIG. 1A with the implant surface depicted as a mesh, according to an embodiment of the invention.

FIG. 2B depicts the mesh layers configuration of the intrasaccular implant from FIG. 1B, according to an embodiment of the invention.

FIG. 3 depicts a cross section of an alternative intrasaccular implant with an invaginated proximal loop design, according to an embodiment of the invention.

FIG. 4 depicts the intrasaccular implant from FIG. 3 with an alternative mechanical release feature, according to an embodiment of the invention.

FIG. 5A depicts a schematic of the delivery system with attached intrasaccular implant inside of a vessel and aneurysm, according to an embodiment of the invention.

FIG. 5B depicts a schematic of the intrasaccular implant deployed in the aneurysm sac while still being attached to the delivery system via the mechanical release mechanism, according to an embodiment of the invention.

FIG. 5C depicts a schematic of the intrasaccular implant deployed in the aneurysm sac after deployment, according to an embodiment of the invention.

FIG. 6A depicts the intrasaccular implant in a partially deployed configuration, with the only the distal loop deployed inside of the aneurysm sac, according to an embodiment of the invention.

FIG. 6B depicts the intrasaccular implant in a partially deployed configuration, with only the distal and middle loops deployed inside of the aneurysm sac, according to an embodiment of the invention.

FIG. 6C depicts the intrasaccular implant in a fully deployed configuration, with the distal, middle, and proximal loops deployed inside of the aneurysm sac, according to an embodiment of the invention.

FIG. 7 depicts the intrasaccular implant with inscribed circles around the distal, middle, and proximal loops of the device, to demonstrate an embodiment where the middle loop has a diameter larger than that of either the distal or proximal loops, according to an embodiment of the invention.

FIG. 8A depicts the intrasaccular device deployed in a non-spherical aneurysm, according to an embodiment of the invention.

FIG. 8B depicts the device from FIG. 8A deployed in a spherical aneurysm, with an overlay of allowable movement of the proximal loop in response to compression from the aneurysm sac, according to an embodiment of the invention.

FIG. 8C depicts the device from FIG. 8A in a deployed and unconstrained configuration, to be used as comparison against FIGS. 8A and 8B which are constrained within aneurysm sacs, according to an embodiment of the invention.

FIG. 9A depicts another embodiment where the intrasaccular implant inflection points of the middle and proximal loops are positioned towards the distal end of the device to support wide-neck aneurysms, according to an embodiment of the invention.

FIG. 9B depicts the intrasaccular implant from FIG. 9A in a partially deployed configuration, with the distal and middle loops deployed inside of an aneurysm sac, according to an embodiment of the invention.

FIG. 9C depicts the intrasaccular implant from FIG. 9A in a partially deployed configuration, with the distal and middle loops deployed inside an aneurysm sac, having the distal loop in contact with the distal wall of the aneurysm sac, according to an embodiment of the invention.

FIG. 9D depicts the intrasaccular implant from FIG. 9A in a fully deployed configuration, with the distal, middle, and proximal loops deployed inside a wide-neck aneurysm sac, according to an embodiment of the invention.

FIG. 10A depicts a separate embodiment of the intrasaccular implant comprising two-loops, a distal loop and a proximal loop, according to an embodiment of the invention.

FIG. 10B depicts the intrasaccular implant from FIG. 10A deployed in a non-spherical aneurysm sac, according to an embodiment of the invention.

FIG. 11A depicts a section view of an intrasaccular implant with ten layers wire mesh between the proximal end of the device and the distal end of the device deployed in an aneurysm sac, according to an embodiment of the invention.

FIG. 11B depicts a section view of an intrasaccular implant with fourteen layers of wire mesh between the proximal end of the device and the distal end of the device deployed in an aneurysm sac, according to an embodiment of the invention.

FIG. 11C depicts a section view of an intrasaccular implant with six layers of wire mesh between the proximal end of the device and the distal end of the device deployed in an aneurysm sac, according to an embodiment of the invention.

FIG. 11D depicts a table describing the number of wire mesh layers in each of the device configurations described in prior figures, and how that correlated to the number of wires providing resistance to blood flow depending on the number of wires used to braid the wire mesh, according to an embodiment of the invention.

FIG. 12A depicts an intrasaccular device in a partially deployed configuration with the distal loop deployed in an angled deployment into a side wall aneurysm, according to an embodiment of the invention.

FIG. 12B depicts an intrasaccular device in a partially deployed configuration with the distal loop and middle loop deployed in an angled deployment into a side wall aneurysm, according to an embodiment of the invention.

FIG. 12C depicts an intrasaccular device in a fully deployed configuration with the distal, middle, and proximal loops deployed in an angled deployment into a side wall aneurysm, with the device attached to a delivery system, according to an embodiment of the invention.

FIG. 12D depicts the mechanical release mechanism of the delivery system from FIG. 12C, with the mechanism engaged at an angle between the delivery system and the intrasaccular implant, according to an embodiment of the invention.

FIG. 13A depicts the intrasaccular device from FIG. 12C with the mechanical release mechanism disengaged, resulting in final deployment of the intrasaccular implant in an aneurysm sac, according to an embodiment of the invention.

FIG. 13B depicts the mechanical release mechanism on the delivery system after disengagement of the intrasaccular implant from the mechanical release mechanism and the proximal end of the intrasaccular implant with the feature connecting the device to the mechanical release mechanism disengaged from the delivery system, according to an embodiment of the invention.

FIG. 13C depicts a cross-section view of the mechanical release mechanism on the delivery system and the proximal end of the intrasaccular implant engaged and connected prior to final delivery where the mechanical release mechanism is at an angle relative to the intrasaccular implant, according to an embodiment of the invention.

FIG. 13D depicts a close-up view of the outer components of the mechanical release mechanism on the delivery system, according to an embodiment of the invention.

FIG. 14A depicts an alternative embodiment of the mechanical release mechanism on the delivery system without an outer sleeve, according to an embodiment of the invention.

FIG. 14B depicts the mechanical release mechanism from FIG. 14A in the engaged configuration, with an outer sleeve holding a tail, where the tail would be attached to the proximal end of an intrasaccular device, according to an embodiment of the invention.

FIG. 14C depicts the mechanical release mechanism from FIG. 14B immediately after disengagement of the mechanical release mechanism from the tail, where the tail is attached to the proximal end of an intrasaccular device, according to an embodiment of the invention.

FIG. 15 depicts the intrasaccular device delivery system, including a pull wire, outer tube, and the device mechanical release mechanism in an engaged configuration without the intrasaccular device attached, according to an embodiment of the invention.

FIG. 16 depicts the intrasaccular device delivery system from FIG. 15 without the proximal finger collar to demonstrate a method of attachment of the delivery system mechanical release mechanism to the delivery system outer tube, according to an embodiment of the invention.

FIG. 17 is a flowchart illustrating an example method of manufacturing an intrasaccular implant, including braiding a mesh body, forming loop structures, assembling a release mechanism, coupling a tail, coupling the release mechanism to a proximal loop, and securing proximal wire ends at the release mechanism.

### DETAILED DESCRIPTION

In the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘including at least A’ includes A as well as the possibility of one or more other additional elements besides A. In the same manner, the phrase, ‘including A’ includes A, as well as the possibility of one or more other additional elements besides A. However, the phrase, ‘including only A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “implant”, the word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

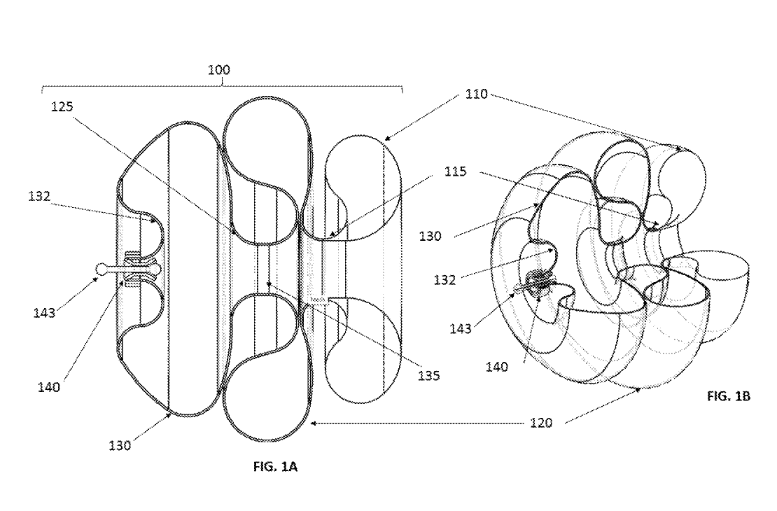

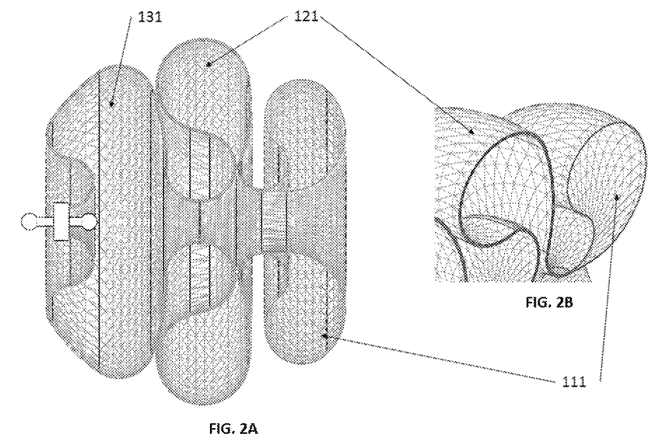

FIG. 1A illustrates a cross section of an intrasaccular device 100 (also referred to hereinafter as intrasaccular implant, intrasaccular implant device, or implant). The intrasaccular device is formed from a plurality of overlapping super elastic wires forming a dense wire mesh. In some embodiments, the intrasaccular device 100 is comprised of three looping patterns formed from the wire mesh, forming a distal loop portion including a distal loop 110, a middle loop portion including a middle loop 120, and a proximal loop portion including a proximal loop 130 in a pattern similar to a series of stacked concentric tori. The intrasaccular device 100 contains a distal neck 115 which connects the distal loop 110 to the middle loop 120, a middle neck 125 which connects the middle loop 120 to the proximal loop 130, and an internal proximal loop 132 which connects the proximal loop 130 to the device release mechanism 140. The distal loop 110, middle loop 120, and proximal loop 130 form a spherical shape when combined, such that the arc path created by each loop continues around each loop to form a circle when viewed in cross-section. The distal loop 110, middle loop 120, proximal loop 130, distal neck 115, middle neck 125, and internal proximal loop 132 are concentric with respect to each other, and share a common central axis. The intrasaccular device 100 may be envisioned as a looping pattern revolved around this common central axis.

In some embodiments of the invention, the height of the distal loop 110 is equal to the height of the distal neck 115 such that the distal neck 115 is completely enveloped by the distal loop 110. In a similar embodiment, the height of the middle loop 120 is equal to the height of the middle neck 125 such that the middle neck 125 is completely enveloped by the middle loop 120.

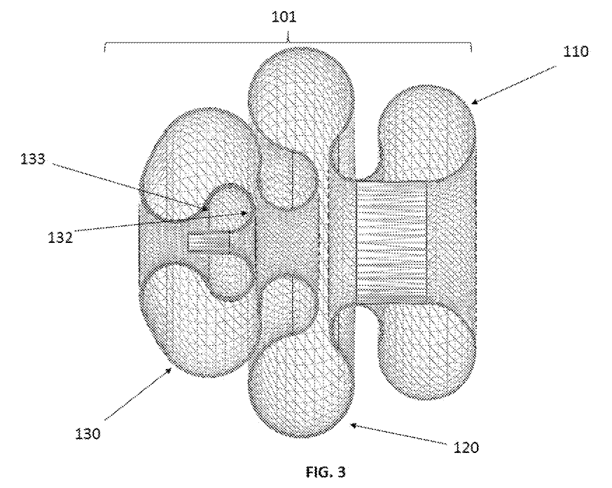

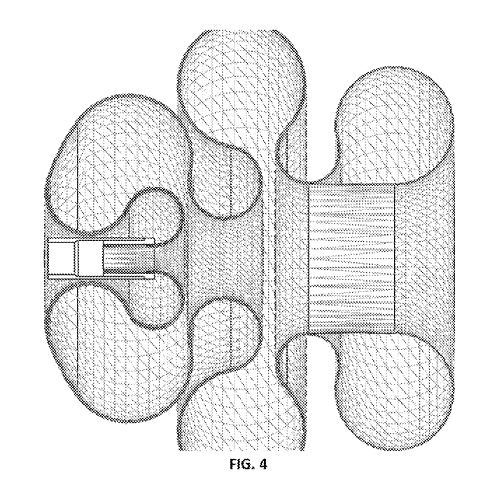

In alternative embodiments of the invention, the internal proximal loop 132 is considerably lengthened to form an invaginated loop 133 resulting in the invaginated loop intrasaccular device 101 as shown in FIG. 3.

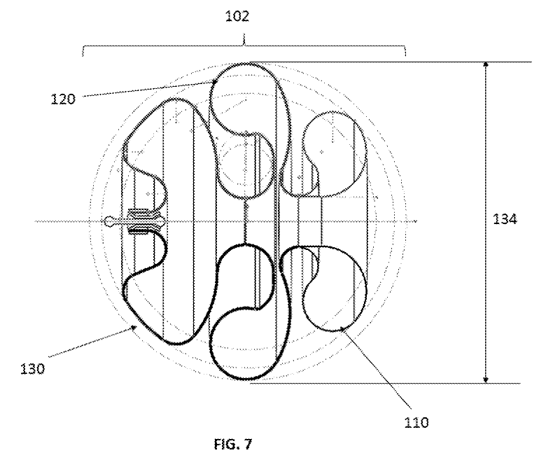

In another embodiment of the invention, the middle loop 120 has a larger diameter than the proximal loop 130 or the distal loop 110, such that the wide middle loop intrasaccular device 102 is not perfectly spherical as shown in FIG. 7.

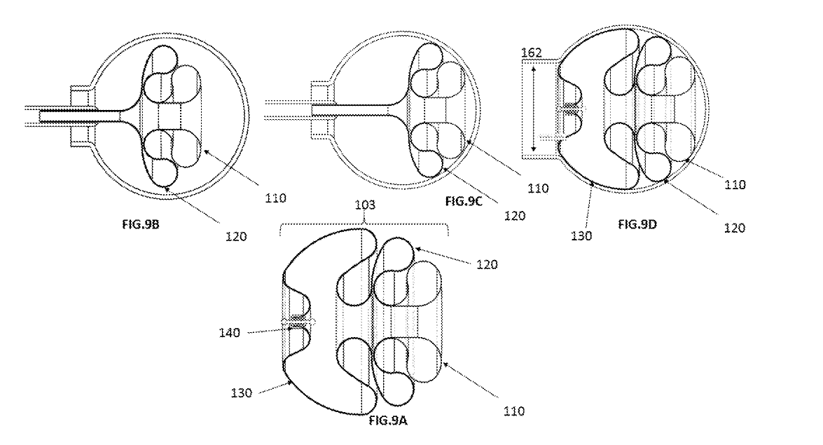

In another embodiment of the invention, the proximal loop 130 and/or middle loop 120 may have an inflection point that is positioned closer to the distal end of the device as shown in FIG. 9A such that the diameter of the middle loop 120 is less than the diameter of the proximal loop 130. In this configuration, the smaller middle loop intrasaccular device 103 is not perfectly spherical.

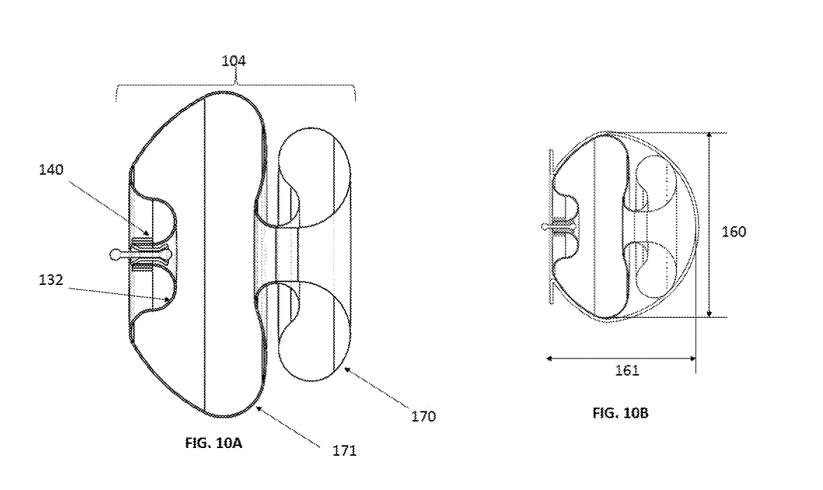

In some embodiments of the invention, the intrasaccular device 100 is comprised of two looping patterns to form the two-loop intrasaccular device 104 as shown in FIG. 10A. The two-loop intrasaccular device 104 is comprised of a distal loop 170 and a proximal loop 171, an internal proximal loop 132, and a device release mechanism 140. In the two-loop intrasaccular device 104, the proximal loop 171 acts as a combination of the proximal loop 130 and middle loop 120 of the three-loop intrasaccular device 100. The two-loop intrasaccular device 104 is intended for smaller aneurysms, where integration of a third loop may not be possible in the limited space available in the aneurysm sac 211.

The distal loop 110 is formed of a plurality of wires formed into a torus shape. The distal loop mesh 111 is comprised of a single layer of a plurality of wires, where each of the external edges of the torus are formed of a singular pattern of overlapping wires. The middle loop 120 and the proximal loop 130 are also formed of a plurality of wires formed into torus shapes. However, the middle loop mesh 121 and the proximal loop mesh 131 are each comprised of a double layer of a plurality of wires, where each of the external edges of the torus are formed of a set of two overlapping patterns of wires, effectively doubling the density of the wire mesh in both the proximal loop 130 and middle loop 120 regions of the intrasaccular device 100.

Density of the wire mesh is of critical importance for effectiveness of the device. The primary course of treatment for any aneurysm is through a flow diversion action, where blood flow into the aneurysm sac is significantly attenuated or eliminated. With flow diversion, blood flow will instead continue down the originally intended blood vessel, without a divergence of blood entering into the aneurysm sac. If blood flow continues to enter into the aneurysm sac unimpeded, there is increasing risk of aneurysm rupture, especially at the distal wall of the aneurysm, which is typically the thinnest and weakest section of the aneurysm. The overall intent to improve efficacy of the device after implantation, is to maximize the overall resistance to blood flow, therefore maximizing the flow diversion properties of the device. Effectively, the more material that is present within the aneurysm sac, the more resistance to blood flow is available. This becomes increasingly important towards the proximal end of the device at the aneurysm neck, where the proximal end of the aneurysm sac meets the parent vessel. Therefore, forming the proximal loop 130 and middle loop 120 using double layers of wires to form the proximal loop mesh 131 and middle loop mesh 121 effectively doubles the flow diversion properties of the device, substantially attenuating flow into the aneurysm.

In some embodiments of the invention, the two layers in the middle loop mesh 121 are offset from one another to ensure that the wires have minimal overlap and the overall amount of coverage generated by the wires is maximized when viewing the device in a cross-sectional planar view parallel to the aneurysm neck.

In some embodiments of the invention, the two layers in the proximal loop mesh 131 are offset from one another to ensure that the wires have minimal overlap and the overall amount of coverage generated by the wires is maximized when viewing the device in a cross-sectional planar view parallel to the aneurysm neck.

In some embodiments of the invention, each individual layer of the mesh for the distal loop mesh 111, middle loop mesh 121, and proximal loop mesh 131 are formed of a plurality of braided wires, where the number of wires may range from a minimum of 24 to over 96 wires, depending on the capabilities of the braiding equipment. As discussed above, additional wires improve overall flow diversion capabilities, so there is a significant benefit to including additional wires into the plurality of braided wires. However, the introduction of additional wires increases the device outer diameter and decreases overall flexibility when in an undeployed configuration prior to implantation. Given that neurovascular aneurysms are of significant importance for occlusive devices, the overall device outer diameter should be minimized and the overall device flexibility should be maximized. There is an inherent balance that should be maintained to ensure that the maximum number of wires are present in the mesh to promote closure of the aneurysm while ensuring that the device can reach the intended treatment location. Neurovascular anatomy is typically comprised of small, tortuous vessels which are difficult to traverse. Therefore, simply maximizing the number of wires in the wire mesh is a sub-optimal approach as it may prevent treatment in smaller or more difficult to access vessels. The double layers in the proximal loop mesh 131 and the middle loop mesh 121 increase the overall amount of material at any given location without the need to increase the number of wires in the mesh, effectively minimizing the constrained device’s outer diameter and maintaining flexibility in the device to improve the ability of the interventionalist to implant the device.

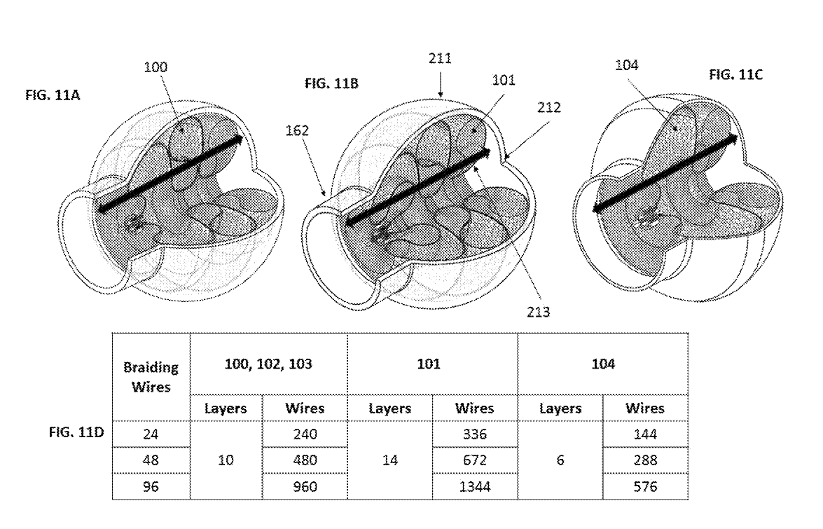

FIG. 11A presents a schematic of the intrasaccular device 100 implanted in an aneurysm sac 211, with an aneurysm neck 162 and undergoing pulsatile blood flow 213. In this configuration, it can be seen that pulsatile blood flow 213 passes through the entirety of the device to reach the distal wall 212 of the aneurysm 211. As pulsatile blood flow 213 works its way through the device, there are multiple layers of resistance due to the layers of wires stacked on top of each other from the proximal loop mesh 131, middle loop mesh 121, and distal loop mesh 111. As such, the resistance to blood flow only ever increases as one proceeds from the aneurysm neck 162 towards the distal section 212 of the aneurysm 211, providing increasing protection for the most sensitive part of the anatomy.

FIGS. 11B and 11C present similar schematics for the deep invaginated loop intrasaccular device 101 and the two-loop intrasaccular device 104 respectively. FIG. 11D presents a summary of the number of effective layers of wire braid that the distal end 212 of the aneurysm 211 has layered between the aneurysm neck 162 and the distal end 212 of the aneurysm 211. For example, the intrasaccular device 100 creates ten effective layers of wire braid between the aneurysm neck 162 and the distal end 212 of the aneurysm 211. Each side of the torus generated by the proximal loop 130 provides two layers of wire braid due to the overlapping wire braid configuration as discussed above, resulting in a total of four layers from the proximal loop 130. Similarly, the torus generated by the middle loop 120 provides two layers due to the overlapping wire braids as discussed above, resulting in an additional four layers from the middle loop 120. Finally, the distal loop 110 creates a single layer on each side of the torus, providing an additional two layers of wire braid from the distal loop 110 for a grand total of 10 layers of wire braid between the aneurysm neck 162 and the distal end 212 of the aneurysm 211. As you increase the number of braiding wires in the device, the total number of wires present to resist blood flow increases proportionally. Therefore, for a 24-wire braid, there is a resultant 240 wire barrier to flow within any vertically oriented path from the aneurysm neck 162 to the distal end 212 of the aneurysm 211. This scales proportionally with the number of wires, such that for a 48-wire braid, there are 480 effective wires, for a 96-wire braid, there are 960 effective wires, and so on. FIG. 11D summarizes similar information regarding the number of total layers and the corresponding number of effective wires for the intrasaccular device 100, invaginated loop intrasaccular device 101, wide middle loop intrasaccular device 102, smaller middle loop intrasaccular device 103, and the two-loop intrasaccular device 104.

In one embodiment of the invention, the diameter of each wire in the wire braid ranges from 0.0008 inches to 0.0012 inches. In an alternative embodiment of the invention, the diameter of the wire may be increased to provide additional resistance to blood flow. In another alternative embodiment of the invention, the wire diameter may be reduced to reduce the constrained diameter and improve flexibility. In another embodiment of the invention, multiple wire diameters may be used in the same braid. This may be beneficial if multiple materials are used in the wire braid, such as the integration of radiopaque wire interspersed between super elastic wire in a ratio that does not significantly impact the mechanical characteristics of the device.

The intrasaccular implant 100 is intended to be attached to a delivery system 201 via a device release mechanism 140. The delivery system 201 incorporates a method of constraining the intrasaccular implant 100, such as an introducer sheath. In this constrained configuration, the intrasaccular implant 100 has a constrained length that is equal to the outer path length of the intrasaccular implant 100 in the deployed configuration, and, therefore, has a height that is significantly longer in the constrained configuration than in the deployed configuration.

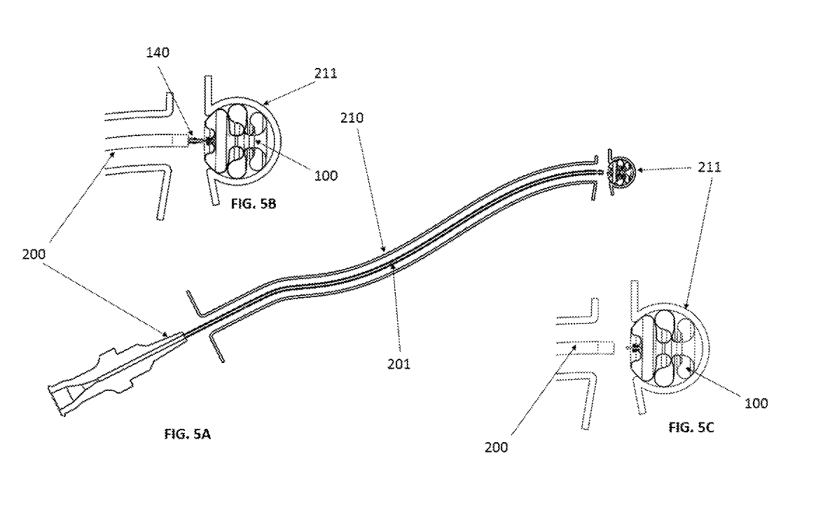

During the procedure, the interventionalist inserts a microcatheter 200 to intended target treatment location. The delivery system 201 with the pre-loaded intrasaccular device 100 in the constrained configuration is advanced forward into and throughout the microcatheter 200 until it reaches the distal end of the microcatheter 200. The intrasaccular device 100 is then advanced out of the distal end of the microcatheter 200 and into the aneurysm sac 211 as shown in FIGS. 5A and 5B. The interventionalist is then able to retract, reposition, and redeploy the intrasaccular device 100 so long as the device release mechanism 140 has not been triggered. Once the interventionalist is satisfied with the position of the intrasaccular device, the device release mechanism 140 is then detached from the intrasaccular device 100 and the delivery system 201 and microcatheter 200 may be removed from the patient, while the intrasaccular device 100 remains in the aneurysm sac 211.

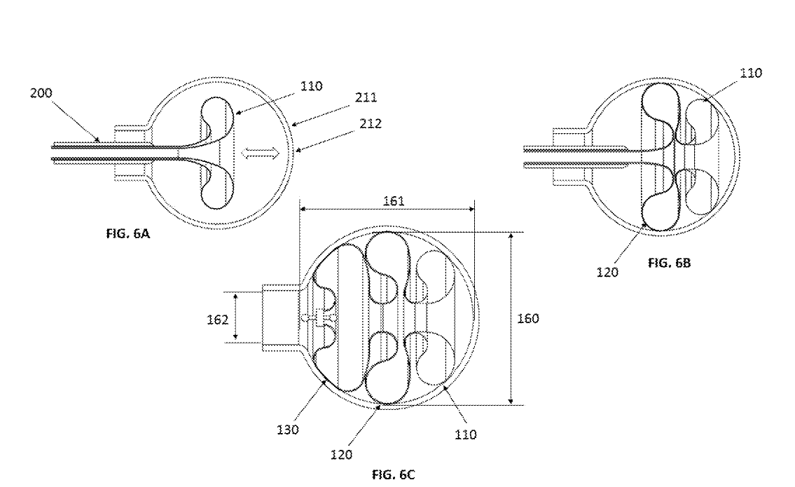

Deployment of the intrasaccular device 100 is performed in four stages. First, the distal loop 110 is advanced out of the microcatheter 200 and into the aneurysm sac 211 as shown in FIG. 6A. In this state the distal loop 110 is free to move within the aneurysm sac based on how the interventionalist advances or retracts the delivery system 201. The distal loop 110 immediately begins to form its shape upon advancement out of the distal end of the microcatheter 200, and provides a wide, circular area of contact against the distal wall of the aneurysm sac 211.

The second step is to advance the middle loop 120 out of the microcatheter 200 and into the aneurysm sac 211 as shown in FIG. 6B. The middle loop 120 takes its full, final torus shape after the entirety of the middle loop 120 has been advanced out of the microcatheter 200. As the middle loop 120 is being advanced out of the microcatheter, it has a substantially cylindrical shape, similar to the constrained or undeployed sections of the intrasaccular device 100. In this partially deployed configuration, the middle loop 120 has a height equal to the cross-sectional path length of the middle loop 120, which is greater than the deployed middle loop height. Once the middle neck 125 has been advanced out of the microcatheter 200, the middle loop 120 foreshortens and immediately takes on its final, deployed torus shape. During this step, the deployed distal loop 110 may be pressed against the distal end of the aneurysm sac 211 in order to provide a sufficient amount of space for the middle loop 120 to be inserted and deployed.

The third step is to advance the proximal loop 130 out of the microcatheter 200 and into the aneurysm sac 211 as shown in FIG. 6C. The proximal loop 130 takes its full, final torus shape after the entirety of the proximal loop 130 has been advanced out of the microcatheter 200. As the proximal loop 130 is being advanced out of the microcatheter, it has a substantially cylindrical shape, similar to the constrained or undeployed sections of the intrasaccular device 100. In this partially deployed configuration, the proximal loop 130 has a height equal to the cross-sectional path length of the proximal loop 130, which is greater than the deployed proximal loop height. Once the internal proximal loop 132 has been advanced out of the microcatheter 200, the proximal loop 130 foreshortens and immediately takes on its final, deployed torus shape. During this step, the deployed distal loop 110 may be pressed against the distal end of the aneurysm sac 211 in order to provide a sufficient amount of space for the proximal loop 130 to be inserted and deployed. Additionally, the middle loop 120 may compress to further distribute the force and permit additional space for the proximal loop 130 to be inserted and deployed.

The fourth and final step is to release the intrasaccular device 100 from the delivery system 201 via the device release mechanism 140. Once the intrasaccular device 100 has been detached from the delivery system 201, the delivery system 201 and the microcatheter 200 are removed from the patient, and the intrasaccular device 100 remains in the aneurysm sac 211 to promote occlusion of the aneurysm.

The segmentation of the intrasaccular device 100 into multiple loops permits each loop to form independently from one another, reducing the amount of device which must be advanced out of the microcatheter 200 prior to the device beginning to take shape and foreshortening. Without this segmentation, the intrasaccular device 100 would remain in its constrained or undeployed configuration, a long cylinder with a diameter approximately equivalent to the microcatheter 200 inner diameter, until all or a majority of the intrasaccular device 100 is advanced out of the microcatheter 200. This acts as a limitation to the amount of material that can be incorporated into the intrasaccular device 100, as there are only two options for deployment of a device which has a height larger than the aneurysm sac 211, both of which are undesired. Option one is to force the device against the distal wall 212 of the aneurysm 211 during deployment, which introduces a risk of damaging the distal wall 212 of the aneurysm 211 and causing a premature rupture. With the distal wall 212 of the aneurysm 211 being the thinnest and therefore weakest part of the aneurysm sac 211, this is the least preferred area to apply force. Additionally, if there was no distal loop 110, the load applied to the distal wall 212 of the aneurysm 211 during deployment would be concentrated in a small area, further increasing the risk of causing a premature rupture. Option two is to advance a portion of the intrasaccular device 100 out of the microcatheter 200 outside of the aneurysm sac 211. This option may cause device misplacement or migration, as it may act unpredictably outside of the aneurysm sac 211. With this option, it is likely that the device will either partially protrude out of the aneurysm sac 211 or fully migrate out of the aneurysm sac 211, both of which cause significant patient risk. The segmentation of the intrasaccular device 100 mitigates the risks in both of these options, by providing a method for incremental and controlled foreshortening of the intrasaccular device 100 during deployment, ensuring that the device can fit inside the aneurysm sac 211 during deployment, and that the amount of force applied to the distal wall 212 of the aneurysm 211 is minimized, both due to the shortening length required to fit inside the aneurysm sac 211, and due to the cushioning effect of the distal loop 110 and the compressibility of the middle loop 120.

The distal loop 110, middle loop 120, and proximal loop 130, all share a similar torus geometry, which loops in an arc away from the centerline of the intrasaccular device 100. This looping and arcing pattern permits each of the loops to be compressed in longitudinal and radial directions. While compressed, the wire mesh compresses, further increasing the density of the wires and overall resistance to blood flow. Additionally, the looping geometry of the distal loop 110 is configured to provide a cushion for deployment of the device into the aneurysm, providing a large contact surface area with the distal wall 212 of the aneurysm 211, therefore dispersing any deployment forces over large areas instead of concentrating and focusing those deployment forces. The middle loop 120 is configured to compress during deployment, further reducing the amount of deployment force that is transferred to the distal loop 110 and therefore the distal wall 212 of the aneurysm 211. Additionally, the middle loop 120 has a diameter that is larger than the proximal loop 130, and is intended to provide radial force against the wall of the aneurysm sac 211. This radial force aids in device retention, minimizes the potential for device migration, and transfers a majority of the forces on the intrasaccular device 100 into the side walls of the aneurysm sac 211 after implantation, further protecting the distal wall 212 of the aneurysm 211 and minimizing the risk of aneurysm rupture.

In some embodiments, the braided mesh body includes a distal loop portion, a proximal loop portion, and an intermediate portion disposed between the distal loop portion and the proximal loop portion. The intermediate portion may include a middle loop portion positioned between the distal loop portion and the proximal loop portion, and may further include one or more neck regions joining adjacent loop portions. During deployment from the catheter, the distal loop portion may begin expanding before the proximal loop portion such that the distal loop portion first defines a widened contact area against the distal wall of the aneurysm sac. The widened contact area may be circumferential or annular in profile and may distribute deployment forces over a larger surface area of the distal wall.

The distal neck 115 and the middle neck 125 have a diameter less than the distal loop 110 and the middle loop 120, however, they are formed from the same wire braid as the rest of the intrasaccular device 100. Therefore, the wire density in both the distal neck 115 and the middle neck 125 is greater than the wire density in the distal loop 110 and the middle loop 120 within each layer. The additional material in both the middle neck 125 and the distal neck 115 add increased stiffness and rigidity, which improves the ability of the intrasaccular device 100 to be advanced out of the microcatheter 200 during delivery and deployment. The stiffness and rigidity of the middle neck 125 and distal neck 115 correlate to the outer diameter of each of those features, with reductions in outer diameter corresponding to increased stiffness and rigidity.

According to some embodiments of the invention, the diameter of the middle loop 120 may be set to a larger diameter than the diameter of the aneurysm sac 211 to further increase the radial force and retention characteristics of the device. The wide middle loop intrasaccular device 102 has a middle loop width 134 that does not share a cylindrical arc with the proximal loop 130 or the distal loop 110 as shown in FIG. 7. In other embodiments of the invention, the diameter of the middle loop 120 is set to approximately the same size as the aneurysm sac 211 to minimize the force applied from the intrasaccular device against the wall of the aneurysm sac 211.

The proximal loop 130 is configured to compress radially against the proximal end of the aneurysm sac 211, provide a termination point for the wire braid, and provide a recessed section to house the device release mechanism 140. The proximal loop 130 creates a looping pattern which is not perfectly circular. This looping pattern is intended to conform to the aneurysm neck 162 and tapers from the outer diameter of the intrasaccular device 100 towards the central axis. The proximal loop 130 contains an internal proximal loop 132 which is recessed within the proximal loop 130 and terminates to the device release mechanism 140. The ends of all of the wire braids terminate into the device release mechanism 140. The internal proximal loop 132 has a depth approximately equal to the length of the device release mechanism 140, such that the device release mechanism 140 does not protrude beyond the proximal end of the intrasaccular device 100 after implantation. Minimization of the protrusion of the device release mechanism 140 reduces the risk of causing embolization in the vessel, as foreign objects protruding into the parent vessel may increase the likelihood of stroke or other embolization related risks.

The device release mechanism 140 is the densest area of the device in terms of raw material, as both ends of the wire braid meet at this location, along with the device release mechanism 140 itself. This provides an area of incredibly high resistance to blood flow, directly in the center of the intrasaccular device 100. This mitigates potential risks imposed by the presence of a central channel 135, which is a narrow cylindrical section that runs through the centerline of the intrasaccular device 100. According to an embodiment of the invention, the central channel 135 does not have an equivalent number of wire mesh layers as the other areas of the device, and is therefore more susceptible to blood flow permeating in the center of the intrasaccular device 100. The device release mechanism 140 provides additional upfront resistance to blood flow into the central channel 135, thereby mitigating that risk. In an alternative embodiment of the invention, the central channel 135 is sufficiently narrow in that the diameter of the middle neck 125 and distal neck 115 outer diameters are minimized to effectively eliminate the central channel 135. In this embodiment, the diameter of the middle neck 125 and the distal neck 115 promotes all wires around the circumference of the cylinder made by these features to be in as close contact as possible to maximize the resistance to blood flow and permits a similar number of wire mesh layers as other areas of the device.

The wire braid initiates and terminates in the same location of the proximal end of the intrasaccular device 100, at the device release mechanism 140. This reduces the need for a second termination point, typically either a ball weld or a collar, which would usually be seen on the distal end of the intrasaccular device 100. This ensures that all features of the intrasaccular device 100 which come in contact with the aneurysm sac 211 are smooth, rounded wire, and no sharp, blunted, or otherwise open ends can come in contact with the aneurysm sac 211, further reducing the risk of causing a premature rupture.

In some embodiments of the invention, the proximal loop 130 has an invaginated loop 133 as a part of the intrasaccular device 101, which further recesses the device release mechanism 140. In this embodiment, the most proximal section of the device release mechanism 140 aligns with or is inside of the invaginated loop 133 such that the device release mechanism 140 does not extend beyond the most proximal portion of the proximal loop 130. In a further embodiment of the invention, the invaginated loop 133 may close off the proximal end of the device such that the device release mechanism 140 is entirely shielded by the wire mesh of the proximal loop 130 and wire mesh of the invaginated loop 133.

In embodiments where there is a two-loop intrasaccular device 104, where the middle loop 120 and the proximal loop 130 are combined, the new proximal loop 171 takes on the characteristics and functionality of both the middle loop 120 and the proximal loop 130 from the three-loop intrasaccular device 100.

In some embodiments of the two-loop intrasaccular device 104, the proximal loop 171 is configured to provide both radial retention within the aneurysm sac 211 and resistance to blood flow at the aneurysm neck 162. The proximal loop 171 may compress against the aneurysm neck region to resist migration of the device 100 while also presenting a dense mesh structure across the aneurysm neck 162 to attenuate blood flow into the aneurysm sac 211.

The diameters and heights of the distal loop 110, middle loop 120, and proximal loop 130, may be modified to accommodate varying aneurysm sizes and shapes, and can range from 2mm in diameter and height to 11mm in diameter and height under typical circumstances. Due to the segmentation of the intrasaccular device 100 into multiple loops, the device is able to be compressed both radially and longitudinally. Therefore, one device size may be able to accommodate a larger range of aneurysm sizes than a non-segmented device.

The segmentation of the intrasaccular device 100 into multiple loops supports the ability of the device to accommodate various shaped aneurysms. Notably, spherical, non-spherical, and wide-neck aneurysms. In all aneurysms, the intrasaccular device undergoes a point of axial reaction 301, where the intrasaccular device 100 is in compression in either the longitudinal or radial direction, and as such modulates shape to conform to the aneurysm sac 211 geometry.

In a spherical aneurysm, such as in FIG. 6C, the aneurysm has an aneurysm width 160 and an aneurysm height 161 which are approximately equal to one another. In a spherical aneurysm, the intrasaccular implant 100 functions as previously described, with the distal loop 110 acting as a cushion during deployment, the middle loop 120 providing radial force to improve retention, and the proximal loop 130 providing stability in the aneurysm neck and initial resistance to blood flow for the aneurysm sac 211.

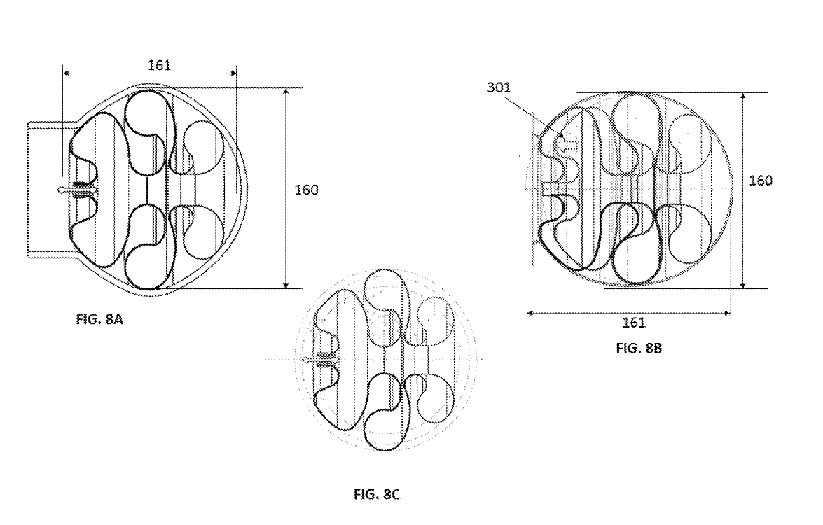

In a non-spherical aneurysm, such as in FIG. 8A the aneurysm has an aneurysm width 160 that is not equal to the aneurysm height 161. In the configuration where the aneurysm width 160 is greater than the aneurysm height 161, the intrasaccular device is compressed in the longitudinal direction, increasing the diameter of the middle loop 120 and proximal loop 130 to more appropriately fit the shape of the aneurysm. In the configuration where the aneurysm width 160 is less than the aneurysm height 161, the intrasaccular device is compressed in the radial direction, which elongates the middle loop 120 and the proximal loop 130 to more appropriately fit the shape of the aneurysm.

In a wide-neck aneurysm, such as in FIG. 9D, the aneurysm may be a spherical or non-spherical aneurysm, but the aneurysm neck 162 may be more similar to the aneurysm width 160 than in typical aneurysms. Wide-neck aneurysms are considered high-risk for treatment, as the possibility of device migration increases as the aneurysm neck 162 diameter approaches the aneurysm width 160. Additionally, wide-neck aneurysms provide additional cross-sectional area for blood to flow into the aneurysm sac 211, further increasing the difficulty in successfully occluding the aneurysm sac 211. In a wide-neck aneurysm, the intrasaccular implant 100 functions similarly to the spherical and non-spherical aneurysms. The distal loop 110 acts as a cushion during deployment, the middle loop 120 provides radial force to improve retention, and the proximal loop 130 provides stability in the aneurysm neck and initial resistance to blood flow for the aneurysm sac 211. The proximal loop 130 expands to meet the aneurysm neck 162, and provide additional retention.

According to an embodiment of the invention, the smaller middle loop intrasaccular device 103 may be used to further improve retention in wide-neck aneurysms. In this embodiment, the smaller middle loop intrasaccular device 103 has a distal loop 110 similar to the intrasaccular device 100, a middle loop 120, and a proximal loop 130, where the middle loop 120 has a diameter less than that of the proximal loop 130. In this embodiment, the middle loop 120 is configured such that the looping pattern is inverted from the intrasaccular device 100, and loops upwards towards the distal end of the device. Similarly, the proximal loop 130 loops upwards towards the distal end of the device. The middle loop 120 extends distally to the proximal end of the distal loop 110. The proximal loop 130 extends distally to the proximal end of the middle loop 120. In this embodiment, the height of the proximal loop 130 is significantly longer than the height of the proximal loop 130 in the intrasaccular device 100. The smaller middle loop 120 and the longer proximal loop 130 provide more area for retention of the proximal loop 130 on the proximal end of the aneurysm sac 211. This is especially important in wide-neck aneurysms, as the increased contact area both reduces the risk of migration, but also reduces the risk of the proximal loop 130 protruding out of the aneurysm neck 162.

Additionally, the smaller middle loop permits the intrasaccular device 103 to deploy both the distal loop 110 and the middle loop 120 fully without contacting the aneurysm sac 211 as shown in FIG. 9B. For this to be possible, the diameter of the middle loop 120 should be less than the aneurysm width 160. In this configuration, both the distal loop 110 and middle loop 120 primarily act as cushions during deployment, and only provide radial retention features once the proximal loop 130 has been fully deployed in the aneurysm sac 211.

According to an embodiment of the invention, the device release mechanism 140 supports a method of mechanical attachment of the intrasaccular device 100 to the delivery system 201. In some embodiments, the device release mechanism 140 connects to the delivery system release mechanism 145. The delivery system 201 includes a pull wire 148, a delivery system outer tube 157, a proximal finger collar 158, a delivery system release mechanism 145, and a sleeve 147. The sleeve 147 is mounted on the distal end of the pull wire 148 and is comprised of a number of sleeve fingers 166 terminating in a sleeve tube 165 on the distal end of the sleeve 147. The sleeve fingers 166 are attached to the pull wire 148. The attachment may utilize welding, adhesives, mechanical fits, or material shaping methods in order to adhere or form the sleeve 147 at the distal end of the pull wire 148. The pull wire 148 and the sleeve 147 are connected such that if the pull wire 148 is retracted, the sleeve 147 retracts an equivalent amount. The sleeve fingers 166 are nested within the negative space created by the distal fingers 146 on the delivery system release mechanism 145. In some embodiments of the invention, there are three distal fingers 146 and three sleeve fingers 166. In alternative embodiments of the invention, more than three distal fingers 146 and sleeve fingers 166 may be incorporated into the design.

A delivery system outer tube 157 is mounted on top of the pull wire 148. The delivery system outer tube 157 terminates to a proximal finger collar 158 on the distal end of the delivery system outer tube 157. The delivery system outer tube 157 inner diameter is equal to or larger than the pull wire 148 outer diameter, such that the pull wire may be forcibly advanced or retracted inside of the delivery system outer tube 157.

The delivery system release mechanism 145 is comprised of distal fingers 146, a flexible pattern 159, and proximal fingers 156. The distal fingers 146 have a distal finger angle 151 which tapers the outer diameter of the delivery system release mechanism 145 such that the distal fingers 146 come in contact with one another when not otherwise forcibly opened. The distal fingers 146 each have distal finger nest holes 152 on the distal end of the distal fingers 146. The delivery system release mechanism 145 has an inner diameter that is greater than or equal to the pull wire 148 outer diameter, such that the pull wire can be forcibly moved inside of the delivery system release mechanism 145.

The delivery system release mechanism 145 is forcibly attached to the delivery system outer tube 157. In an embodiment of the invention, the delivery system release mechanism 145 includes finger tabs 153 at the proximal end of the proximal fingers 156. In this embodiment, the delivery system outer tube 157 contains slots on the distal end of the delivery system outer tube 157 which are the negative of the finger tabs 153. This permits the finger tabs 153 to fit into the slots on the delivery system outer tube 157. Additional methods of adherence may be used in conjunction with the finger tabs 153, such as welding, adhesives, or compression fittings. In an embodiment of the invention, the finger tabs 153 are adhered to the delivery system outer tube 157 via a proximal finger collar 158. The proximal finger collar 158 rests over the finger tabs 153 after they have been engaged with the delivery system outer tube 157. As the pull wire 148 is on the inner diameter, this effectively locks the proximal fingers 156 in place. In an alternative embodiment, the delivery system release mechanism 145 is attached to the delivery system outer tube 157 via alternative methods, such as adhesives, hooks, coils, or welding.

The finger tabs 153 on the proximal fingers 156 ensure that the delivery system outer tube 157 is rigidly connected to the delivery system release mechanism 145. This effectively creates a two-body system, where there is the pull wire 148 and sleeve 147 acting as a single body, while the delivery system outer tube 157 and delivery system release mechanism 145 act as the second body.

The delivery system release mechanism 145 may include a flexible pattern 159, which is an interrupted spiral laser cut pattern intended to provide flexibility to that region of the delivery system. The flexible pattern 159 may modulate the width of the laser cuts, the frequency of the laser cuts, or the spacing of the laser cuts which will each have an impact on the overall flexibility of the component. The more cuts present, the closer together the laser cuts, or the narrower the width of the cut segments (wider the width of the cuts/kerf in the flexible pattern 159), the more flexible that region will be, especially compared to solid tubing.

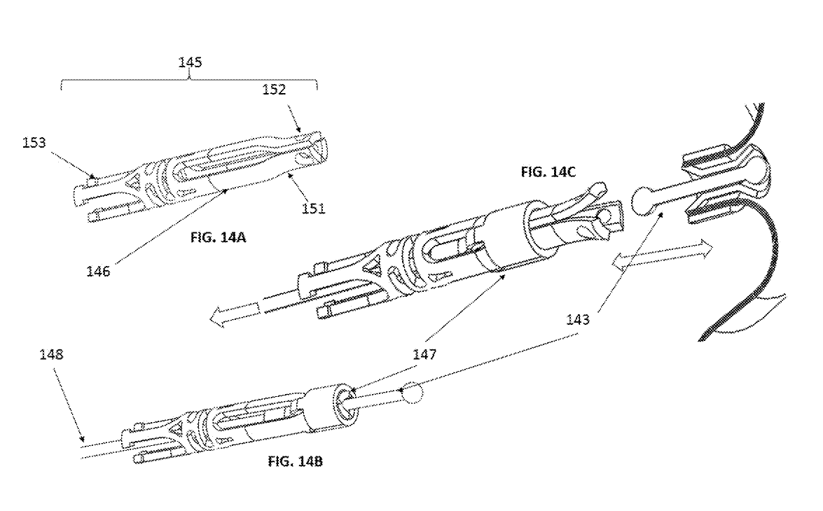

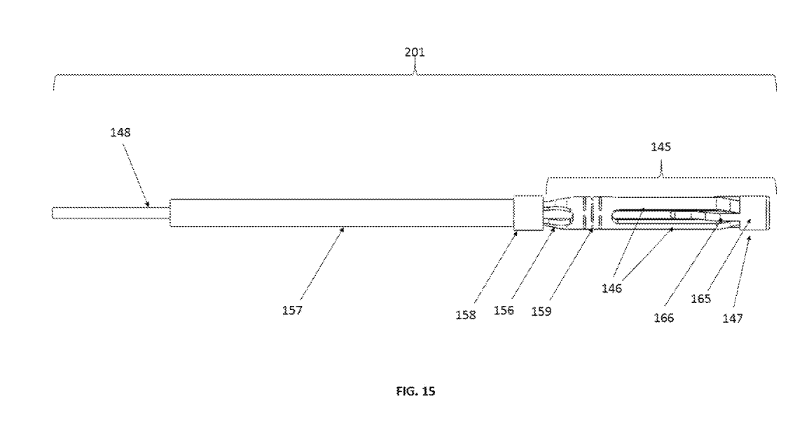

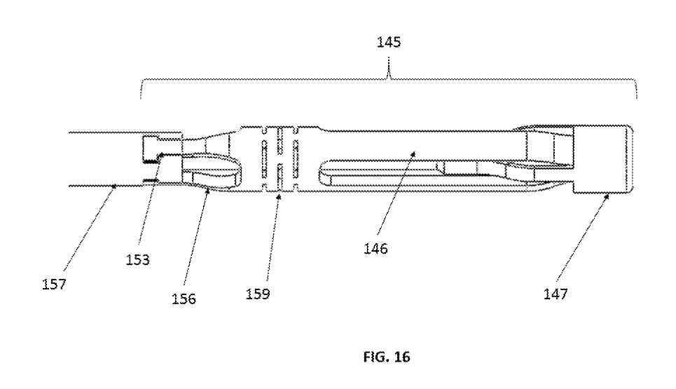

Once assembled, the two-body system can be configured in either an engaged configuration, such as in FIG. 14B, or a disengaged configuration, such as in FIG. 14C. In the engaged configuration, the sleeve 147 sits over the distal end of the distal fingers 146, specifically over the distal fingers nest holes 152. In this configuration, the sleeve 147 compresses the distal fingers 146, forcing the distal fingers 146 to apply pressure concentrically towards the centerline of the delivery system. To modify the delivery system from the engaged configuration to the disengaged configuration, as would be done during final deployment and implantation of the intrasaccular implant 100, the pull wire 148 would be retracted, while the delivery system release mechanism 145 maintains its position. This results in the sleeve 147 moving proximally over the distal fingers 146. The sleeve fingers 166 slide along the slots created by the distal fingers 146, permitting the sleeve 147 to slide proximally and expose the distal finger angle 151 and distal finger nest holes 152.

According to one embodiment of the invention, the delivery system release mechanism 145 is formed of a shape memory alloy such as nitinol, and the designated shape of the distal fingers 146 are in a splayed-out configuration as seen in FIG. 14C, such that the inner diameter at the distal end of the distal fingers 146 is greater than the inner diameter of the rest of the delivery system release mechanism 145. The sleeve 147 restricts this shape and compresses the distal fingers 146 until the sleeve 147 is removed and the original intended shape of the distal fingers 146 can be realized.

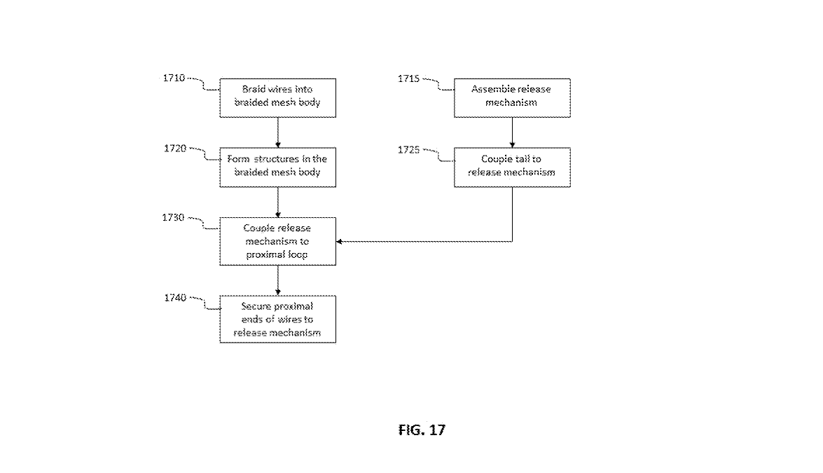

FIG. 17 depicts a flowchart of an example method of manufacturing the intrasaccular implant 100. In some embodiments, the method includes braiding wires into a braided mesh body at step 1710 and forming structures in the braided mesh body at step 1720. The formed structures may include one or more loop portions of the intrasaccular implant 100, including a distal loop, a middle loop, and a proximal loop, and may further include an internal proximal loop configured to receive or support the device release mechanism 140.

In some embodiments, the method further includes assembling the release mechanism at step 1715 and coupling a tail to the release mechanism at step 1725. The tail may be coupled to the release mechanism before the release mechanism is attached to the braided mesh body. After formation of the braided mesh body and assembly of the release mechanism, the method may include coupling the release mechanism to the proximal loop at step 1730 such that the release mechanism is positioned at a proximal region of the intrasaccular implant 100.

In some embodiments, the method further includes securing proximal ends of the wires to the release mechanism at step 1740. By securing the proximal wire ends at the release mechanism, the braided mesh body may be configured such that braid terminations are localized at the proximal region of the intrasaccular implant 100 rather than at a distal end of the implant. This arrangement may facilitate formation of a distal loop without a distal braid termination point and may permit the release mechanism to be recessed within the proximal portion of the intrasaccular implant 100.

According to an alternative embodiment of the invention, the inner diameter of the sleeve 147 is equal to or less than the outer diameter of the delivery system release mechanism 145. When the sleeve 147 is retracted by retracting the pull wire 148, the sleeve 147 moves proximally over the distal fingers 146. Due to the compression of the proximal end of the distal fingers 146, the distal ends of the distal fingers 146 naturally splay-out as seen in FIG. 14C, such that the inner diameter at the distal end of the distal fingers 146 is greater than the inner diameter of the rest of the delivery system release mechanism 145.

According to an alternative embodiment of the invention, the delivery system outer tube 157 has a length that is considerably shorter than the pull wire 148. In this embodiment, there is a secondary delivery system outer tube proximally adjacent to the first and formed of an alternative, more flexible material, such as a polymer or a flexible cut geometry metal tube. This would permit the delivery system outer tube 157 to be formed of a rigid material, such as stainless steel or another metal, while permitting the remainder of the delivery system to remain flexible to promote deliverability to the target treatment location.

According to an embodiment of the invention, the delivery system outer tube 157 may include a spiral laser cut geometry to improve the flexibility of the section. According to another embodiment of the invention, the delivery system outer tube 157 may be covered in another tube to provide a consistent outer diameter which aligns closely with the intended microcatheter 200 inner diameter. This outer tube may be formed of a plastic material and may be adhered to the delivery system outer tube 157 via heat shrinking, UV curing, or adhesive methods.

The intrasaccular device 100 includes a device release mechanism 140 at the termination of the proximal loop 130. The device release mechanism 140 is formed of a collar 141, an inner collar 142, a tail 143, and the termination of both ends of the wire braid. The collar 141, inner collar 142, and termination of both ends of the wire braid may be adhered to each other through welding, adhesive, UV curing, or compression fitting. The tail 143 is comprised of a tail proximal ball 149 and a tail distal ball 150 connected by a tail shaft 163. The tail shaft 163 has a diameter less than the diameter of both the tail proximal ball 149 and the tail distal ball 150. The tail shaft 163 has an outer diameter less than the inner diameter of the inner collar 142. The tail proximal ball 149 and tail distal ball 150 have outer diameters greater than the inner diameter of the inner collar, such that the tail 143 cannot be removed from the device release mechanism 140 after it has been assembled.

In an embodiment of the invention, the inner collar 142 is tapered on both the proximal and distal ends, such that the diameter at either end is larger than the diameter in the center. In a further embodiment, the diameter of the flared section of the inner collar 142 is similar or equal to the diameter of the tail proximal ball 149 and/or tail distal ball 150.

The tail 143 is intended to mate with the delivery system release mechanism 145, such that the tail proximal ball 149 fits into the distal fingers 146. The tail proximal ball 149 has a diameter approximately equal to the diameter of the distal finger nest holes 152. When in the engaged configuration, the tail proximal ball 149 rests inside the distal finger nest holes 152, with the sleeve 147 resting on top of the distal finger nest holes 152. This causes the sleeve 147 to compress the distal fingers 146, thereby compressing the tail proximal ball 149 and holding it in place via a compression fit. The tail distal ball 150 outer diameter is larger than the inner collar 142 inner diameter, thereby preventing the tail 143 from being pulled out of the intrasaccular device. While the sleeve 147 maintains its position in this manner, the delivery system release mechanism 145 will be engaged with and unable to separate from the device release mechanism 140, thereby attaching the intrasaccular implant 100 to the delivery system 201.

To deploy or release the intrasaccular device 100, the pull wire 148 may be retracted, at which time the sleeve 147 will be retracted, removing the outer compression on the distal fingers 146. Without the outer compression on the distal fingers 146, the distal fingers 146 will increase in diameter a sufficient amount to permit the tail proximal ball 149 to disengage from the delivery system release mechanism 145, thereby deploying the intrasaccular device 100 in the target treatment location. Until this deployment step is taken, the intrasaccular device 100 may be retracted, repositioned, or removed from the current location. This ensures that the interventionalist can expand the intrasaccular device 100 into the aneurysm sac 211, assess the fit of the intrasaccular device 100, and then make modifications if necessary. Due to variations in aneurysm sac 211 shape and geometry, it is possible that an incorrectly sized intrasaccular device 100 may be expanded into the aneurysm sac 211. If this occurs, the interventionalist has the ability to remove the intrasaccular device 100 so long as it has not been detached from the delivery system 201.

In some embodiments, the delivery system release mechanism 145 is fixed to a distal end region of the delivery system outer tube 157. The tail proximal ball 149 defines a ball feature of the intrasaccular device 100 that is sized to be received within the distal finger nest holes 152. When the sleeve 147 is in the engaged position, the sleeve 147 compresses the distal fingers 146 to retain the ball feature within the nest holes 152, and when the sleeve 147 is retracted to the disengaged position, the distal fingers 146 are permitted to release the ball feature.

According to some embodiments of the invention, a handle or trigger mechanism may be attached to the proximal end of the pull wire 148 to facilitate with controlled device release. The handle or trigger mechanism may permit the advancement of the pull wire 148 and the device system release mechanism 145 in tandem until a time at which the handle or trigger mechanism is engaged, which retracts the pull wire 148 a pre-specified amount, thereby disengaging the intrasaccular device 100 from the delivery system 201.

According to an alternative embodiment of the invention, the device release mechanism 140 may include a non-mechanical detachment mechanism, such as electrolytic, chemical, or heat-based detachment mechanisms.

The simplest aneurysm location for delivery and deployment is when there is a straight approach from a parent vessel, such as in a bifurcation, where the aneurysm is located between the two legs of the bifurcation. Current devices on the market are limited in their ability to treat vessels that do not fall into this situation, as the nature of the devices and the delivery systems require long, straight paths of approach. There are aneurysms which require deployment at an angle relative to the parent vessel. In those cases, if the current devices are unable to reach the intended treatment location, alternative techniques are required to be used.

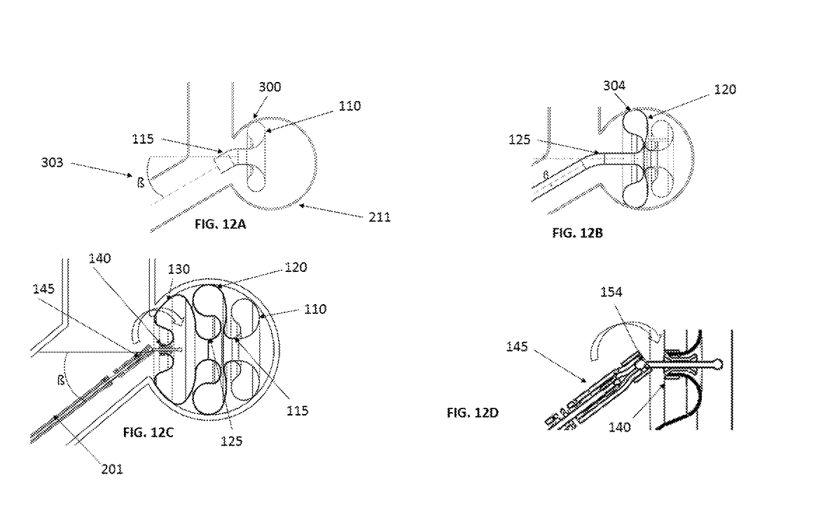

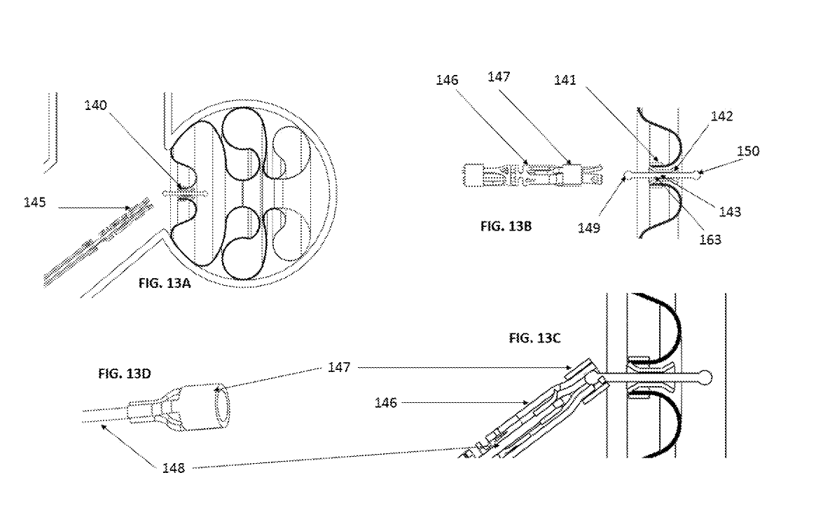

The intrasaccular device 100 and corresponding delivery system 201 is capable of deploying the intrasaccular device 100 in an angled deployment configuration due to both the segmentation of the intrasaccular device 100 into separate looping patterns, and due to the delivery system release mechanism 145 and corresponding device release mechanism 140. As outlined in FIGS. 12A-12D, when approaching an aneurysm with an angle of deployment 303, the basic approach of the device is similar to previous descriptions. As the distal loop 110 is deployed into the aneurysm sac 211, it creates a distal loop contact point 300, which is used as an anchor point to re-orient the deployed sections of the intrasaccular device 100 such that the central axis of the intrasaccular device 100 is parallel to the central axis of the aneurysm sac 211. While the distal loop 110 is deployed, the distal neck 115 acts as a flexible hinge, permitting the deployed distal loop 110 to maintain the angle of deployment 303 relative to the delivery system 201, while still permitting the intrasaccular device 100 to be further advanced into the aneurysm sac 211. As the intrasaccular device 100 is continuously advanced, the middle loop 120 is deployed, creating a middle loop contact point 304. The distal loop contact point 300 disengages, permitting the middle loop 120 to provide additional stability to the intrasaccular device 100 inside the angled aneurysm. Once the middle loop 120 is deployed, the intrasaccular device 100 central axis is colinear with the central axis of the aneurysm sac 211. With the middle loop 120 deployed, the middle neck 125 acts as the hinge point, similar to how the distal neck 115 did prior to the middle loop 120 deployment. The proximal loop 130 may then be advanced and deployed into the aneurysm sac, where its orientation is dictated by the distal loop 110 and middle loop 120 orientations, which had already been aligned to the angle of deployment 303. The ball joint 154 connecting the device release mechanism 140 to the delivery system release mechanism 145 then acts like the hinge, similar to the distal neck 115 and middle neck 125 in previous steps. The hinge effect of the ball joint 154 permits the delivery system 201 to maintain an angle of deployment 303 while remaining engaged to the intrasaccular device 100 without applying any alternative forces which may cause the intrasaccular device 100 to shift inside the aneurysm sac 211. The intrasaccular device 100 may then be deployed as described above, with the ball joint 154 disengaging, separating the device release mechanism 140 from the delivery system release mechanism 145, and fully deploying the intrasaccular device into the angled aneurysm.

According to an embodiment of the invention, the distance between the distal finger nest holes 152 to the distal end of the distal fingers 146 on the delivery system release mechanism 145 is minimized to permit a larger amount of rotation of the ball joint 154. In some embodiments, minimizing the distance between the distal finger nest holes 152 and the distal ends of the distal fingers 146 increases angular articulation between the delivery system 201 and the intrasaccular device 100 while the intrasaccular device remains engaged to the delivery system 201. The reduced distance permits a greater range of angular movement of the ball feature relative to the distal fingers 146 during angled delivery into an aneurysm sac.

According to an embodiment of the invention, the sleeve tube 165 may be partially recessed, have grooves, or have a tapered wall thickness on the distal end of the sleeve tube 165 to allow the tail 143 to swivel inside of the ball joint 154 more easily, thereby permitting larger amounts of rotation and increasing the ability of the device to treat angled aneurysms.

According to an embodiment of the invention, the wire braid includes super elastic wire, such as nitinol, or a combination of super elastic wire and non-super elastic wire. In this configuration, the non-super elastic wire is interspersed throughout the device, such as every fourth wire or every eight wire. Integration of non-super elastic wires may provide opportunities to modify the radial strength, flexibility, or improve radiopaque characteristics of the intrasaccular device 100. The wire braid may be formed of multiple wire braiding styles, such as one over one, two over two, one over two, etc.

The intrasaccular device 100 may include radiopaque elements to improve visibility under fluoroscopy. Radiopaque elements may be introduced in the form of radiopaque core super elastic wires in the wire braid, radiopaque wires in the wire braid, inclusion of radiopaque materials in the device release mechanism 140 or as part of a collar adjacent to the device release mechanism 140, or radiopaque coatings on the intrasaccular device. Additionally, the delivery system 201 may include radiopaque elements similar, such as collars, marker bands, or coils integrated into the delivery system such as the delivery system release mechanism 145. In some embodiments of the invention, the radiopaque elements, such as collars, marker bands, or coils may be positioned on the middle neck 125 and the distal neck 115. In these embodiments, the radiopaque element may serve as a visible indicator for the interventionalist as to when each of the loops has been advanced out of the delivery catheter.

In some embodiments of the invention, the intrasaccular device 100 can be manufactured by braiding a plurality of wires to form a wire braid and then forming the wire braid into the desired loop geometry of the device. In some embodiments, the wire braid is formed to define the distal loop 110, the distal neck 115, the middle loop 120, the middle neck 125, the proximal loop 130, and the internal proximal loop 132 about a common central axis. In embodiments of the two-loop intrasaccular device 104, the wire braid may instead be formed to define the distal loop 170, the proximal loop 171, and the internal proximal loop 132. In some embodiments, the wire braid is manufactured such that the braid initiates and terminates at the proximal end of the device at the device release mechanism 140, thereby eliminating a separate distal braid termination point.

In some embodiments of the invention, the braided mesh body is formed such that proximal ends of the plurality of wires are secured at the device release mechanism 140 coupled to the internal proximal loop 132. In this manner, the braid initiates and terminates at the proximal end of the intrasaccular device 100, thereby eliminating a distal braid termination point and localizing braid terminations within the recessed proximal region.

In some embodiments of the invention, each mesh layer of the intrasaccular device 100 may be formed from a plurality of braided wires, where the number of wires may range from 24 to over 96 wires depending on the capabilities of the braiding equipment. In some embodiments, the wire diameter ranges from 0.0008 inches to 0.0012 inches, although larger or smaller diameters may be used to modify flow resistance, constrained profile, or flexibility. In some embodiments, the wire braid includes super elastic wire, such as nitinol, alone or in combination with non-super elastic wire. In some embodiments, non-super elastic wire or radiopaque wire may be interspersed within the wire braid, such as one wire out of four or one wire out of eight. The wire braid may be formed using one-over-one, two-over-two, one-over-two, or other braiding styles.

In some embodiments of the invention, manufacture of the intrasaccular device 100 includes forming the distal loop mesh 111 as a single layer and forming the middle loop mesh 121 and the proximal loop mesh 131 as double layers to increase material density toward the proximal end of the device. In some embodiments, the two layers of the middle loop mesh 121 and or the proximal loop mesh 131 are offset relative to one another to minimize overlap of wires and increase coverage when viewed in a cross-sectional planar view parallel to the aneurysm neck. The diameters and heights of the loops may be selected to accommodate different aneurysm sizes and shapes, and in some embodiments range from 2 mm to 11 mm.

In some embodiments of the invention, manufacture of the device release mechanism 140 includes coupling the collar 141, the inner collar 142, and the terminations of both ends of the wire braid to each other by welding, adhesive, UV curing, or compression fitting. The tail 143, including the tail proximal ball 149, the tail distal ball 150, and the tail shaft 163, may be assembled with the inner collar 142 such that the tail shaft 163 extends through the inner collar 142 and the tail proximal ball 149 and the tail distal ball 150 are larger than the inner diameter of the inner collar 142, thereby retaining the tail 143 within the device release mechanism 140. In some embodiments, the inner collar 142 is tapered on the proximal and distal ends.

Features discussed above which reference the intrasaccular device 100 may also be incorporated into the alternative embodiments of the intrasaccular device 100 previously discussed.

1. An intrasaccular implant for vascular occlusion of an aneurysm, the implant comprising:
a self-expanding braided mesh body configured for constrained delivery through a catheter and expansion within an aneurysm sac,
wherein the braided mesh body includes a distal loop portion configured to contact a distal wall of the aneurysm sac over a widened contact area during deployment, a proximal loop portion configured to be positioned adjacent an aneurysm neck, and an intermediate portion between the distal loop portion and the proximal loop portion,
wherein the braided mesh body is configured such that, during deployment from the catheter, the distal loop portion expands before the proximal loop portion and the braided mesh body foreshortens in stages as successive loop portions expand, and
wherein the proximal loop portion defines a recessed proximal region configured to house a release mechanism such that the release mechanism does not protrude beyond a proximal end of the implant after deployment.

2. The implant of claim 1,
wherein the distal loop portion includes a distal loop, the middle loop portion includes a middle loop, and the proximal loop portion includes a proximal loop, and
wherein the distal loop, the middle loop, and the proximal loop are connected in series by a distal neck and a middle neck.

3. The implant of claim 2, wherein the distal loop has a single mesh layer and each of the middle loop and the proximal loop has a double mesh layer.

4. The implant of claim 3, wherein the double mesh layer of at least one of the middle loop or the proximal loop comprises two offset layers arranged to reduce wire overlap in a cross-sectional view parallel to the aneurysm neck.

5. The implant of claim 2, wherein the middle loop has a diameter greater than each of a diameter of the distal loop and a diameter of the proximal loop, to increase radial retention within the aneurysm sac.

6. The implant of claim 2, wherein the middle loop has a diameter less than a diameter of the proximal loop and the proximal loop extends distally beyond a proximal end of the middle loop to increase retention in a wide-neck aneurysm.

7. The implant of claim 2, wherein the proximal loop includes an invaginated loop that further recesses the release mechanism within the implant.

8. The implant of claim 1, wherein a wire braid forming the braided mesh body initiates and terminates at the recessed proximal region that houses the release mechanism, such that the implant is free of a distal braid termination point.

9. The implant of claim 1,
wherein the braided mesh body includes two loops, a distal loop and a proximal loop, and
wherein the proximal loop is configured to provide both radial retention within the aneurysm sac and resistance to blood flow at the aneurysm neck.

10. A delivery system for delivering an intrasaccular implant into an aneurysm sac, the delivery system comprising:
an outer tube;
a pull wire movable relative to the outer tube;
a release mechanism fixed to a distal end region of the outer tube and including distal fingers having nest holes sized to receive a ball feature of the intrasaccular implant; and
a sleeve coupled to the pull wire and movable relative to the distal fingers between an engaged position in which the sleeve compresses the distal fingers to retain the ball feature in the nest holes and a disengaged position in which the sleeve is retracted to permit the distal fingers to release the ball feature.

11. The delivery system of claim 10, wherein the release mechanism further includes:
proximal fingers having finger tabs received in slots of the outer tube; and
a proximal finger collar disposed over the finger tabs to lock the release mechanism to the outer tube.

12. The delivery system of claim 10, wherein the release mechanism includes a flexible pattern between the distal fingers and proximal fingers to increase flexibility of the delivery system.

13. The delivery system of claim 10, wherein the distal fingers are formed of a shape memory alloy and are biased toward a splayed configuration when the sleeve is in the disengaged position.

14. The delivery system of claim 10, wherein the release mechanism comprises three distal fingers and the sleeve comprises three sleeve fingers nested with the distal fingers.

15. The delivery system of claim 10, wherein a distance between the nest holes and distal ends of the distal fingers is minimized to increase angular articulation between the delivery system and the intrasaccular implant while the intrasaccular implant remains engaged to the delivery system.

16. A method of manufacturing an intrasaccular implant, the method comprising:
braiding a plurality of wires into a braided mesh body;
forming the braided mesh body into a distal loop, a middle loop, a proximal loop, a distal neck between the distal loop and the middle loop, a middle neck between the middle loop and the proximal loop, and an internal proximal loop recessed within the proximal loop; and
securing proximal ends of the plurality of wires at a device release mechanism coupled to the internal proximal loop such that the plurality of wires initiates and terminates at a proximal end of the intrasaccular implant.

17. The method of claim 16, wherein forming the braided mesh body comprises forming the distal loop as a single-layer mesh and forming each of the middle loop and the proximal loop as a double-layer mesh.

18. The method of claim 17, wherein forming at least one of the middle loop or the proximal loop as the double-layer mesh comprises offsetting two wire layers relative to one another to reduce wire overlap in a cross-sectional planar view parallel to an aneurysm neck.

19. The method of claim 16, wherein braiding the plurality of wires comprises braiding from 24 to 96 wires, the wires having diameters ranging from 0.0008 inches to 0.0012 inches.

20. The method of claim 16, further comprising:
assembling the device release mechanism by adhering a collar, an inner collar, and terminations of both ends of the braided mesh body to each other by welding, adhesive, UV curing, or compression fitting; and
coupling a tail comprising a proximal ball, a distal ball, and a shaft extending therebetween to the device release mechanism such that the shaft extends through the inner collar and the proximal ball and the distal ball are retained against removal from the inner collar.
